(19) United States
(12) Patent Application Publication
(10) Pub. No.: US 2010/0131001 A1
(43) Pub. Date: May 27, 2010

(54) Targeted Drug Delivery for Aneurysm Treatment

(75) Inventor: Susan Rea Peterson, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/276,589

(22) Filed: Nov. 24, 2008

(51) Int. Cl. A61M 29/00; A61B 19/00

(52) U.S. Cl. 606/200; 128/898

(57) ABSTRACT

Disclosed herein are systems and methods related to aneurysm treatment. More specifically, the systems and methods disclosed herein relate to localized treatment of aneurysms utilizing polymeric micelles that release therapeutic agent(s) when exposed to energy.

### FIELD OF THE INVENTION

Disclosed herein are systems and methods related to aneurysm treatment. More specifically, the systems and methods disclosed herein relate to localized treatment of aneurysms utilizing polymeric micelles that release therapeutic agent(s) when exposed to energy.

### BACKGROUND OF THE INVENTION

An aneurysm is a localized dilation of a blood vessel usually caused by degeneration of the vessel wall. These weakened sections of vessel walls can burst, causing an estimated 32,000 deaths in the United States each year. Additionally, aneurysm deaths are suspected of being underreported because sudden unexplained deaths, about 450,000 in the United States alone, are often simply misdiagnosed as heart attacks or strokes while many of them may be due to aneurysms.

US surgeons treat approximately 50,000 abdominal aortic aneurysms each year, typically by replacing the abnormal section of vessel with a polymer graft in an open surgical procedure. A less-invasive procedure that has more recently been used is the placement of a stent graft at the aneurysm site. Stent grafts are tubular devices that span the aneurysm site to provide support without replacing a section of the vessel. The stent graft, when placed within a vessel at an aneurysm site, acts as a barrier between blood flow and the weakened wall of a vessel, thereby decreasing pressure on the damaged portion of the vessel. Patients whose multiple medical comorbidities make them very high risk for conventional aneurysm repair can be candidates for stent grafting.

Despite the effectiveness of stent grafting, once the aneurysmal site is bypassed, the aneurysm remains. The aneurysmal tissue can continue to degenerate such that the aneurysm continues to increase in size due to the continued thinning of the vessel wall. Thus, there is a need in the art to treat aneurysms themselves and/or to slow or stop continued aneurysm growth following stent graft placement. Moreover, it would be desirable to provide treatments to the aneurysm that can be controlled even following placement at the aneurysm site. The present invention provides such advances.

### SUMMARY OF THE INVENTION

Embodiments according to the present invention provide methods, stent grafts, and treatment kits that can be used to controllably treat aneurysms following stent graft deployment.

One method disclosed herein includes a method of treating an aneurysm comprising: delivering a stent graft to the site of the aneurysm; deploying the stent graft to span the aneurysm; locally administering polymeric micelles containing at least one therapeutic agent to the site of the aneurysm; and exposing the polymeric micelles to sufficient energy to affect the release of the at least one therapeutic agent from the polymeric micelles.

In another embodiment of the methods disclosed herein, the energy is selected from the group consisting of ultrasonic energy, radiofrequency (RF) energy, ultraviolet (UV) energy, infrared (IR) energy, visible light, and combinations thereof.

In another embodiment of the methods disclosed herein, the locally administering comprises: applying the polymeric micelles to the outer surface of the stent graft and/or incorporating the polymeric micelles into a coating on the stent graft.

In another embodiment of the methods disclosed herein, the locally administering comprises: incorporating the polymeric micelles into a coating and placing the coating on the outer surface of the stent graft.

In another embodiment of the methods disclosed herein, the locally administering comprises: attaching a delivery device to the stent graft wherein the delivery device holds and releases the polymeric micelles.

In another embodiment of the methods disclosed herein, the delivery device is a pouch.

In another embodiment of the methods disclosed herein, the locally administering comprises: providing a stent graft with two layers; partially adhering the layers together so that pouches are formed; loading the pouches with polymeric micelles containing at least one therapeutic agent; wherein following deployment of the stent graft, the first layer is exposed to blood flow and the second layer faces the blood vessel wall and is semi-permeable.

In another embodiment of the methods disclosed herein, the locally administering comprises: applying the polymeric micelles directly to the outer surface of the stent graft while the stent graft is compressed within a stent graft deployment catheter.

In another embodiment of the methods disclosed herein, the locally administering comprises: administering the polymeric micelles through a delivery catheter and/or an injection catheter.

In another embodiment of the methods disclosed herein, the polymeric micelles substantially fill the aneurysm sac.

In another embodiment of the methods disclosed herein, the injection catheter is selected from the group consisting of a single lumen injection catheter and a multilumen injection catheter.

In another embodiment of the methods disclosed herein, the polymeric micelles are administered through at least two injection catheters wherein the first and second injection catheters reach the aneurysm site through a different route.

Stent grafts are also disclosed herein. In one embodiment of the stent grafts disclosed herein, the stent graft comprises polymeric micelles that contain at least one therapeutic agent and release the at least one therapeutic agent when exposed to sufficient energy wherein the polymeric micelles are one or more of applied to the outer surface of the stent graft, incorporated within a coating applied to the stent graft or within a delivery device associated with the stent graft.

In another embodiment of the stent grafts disclosed herein, the stent graft comprises polymeric micelles incorporated within a coating applied to the stent graft wherein the coating is biodegradable.

In another embodiment of the stent grafts disclosed herein, the stent graft comprises polymeric micelles incorporated within a coating applied to the stent graft wherein the coating is temperature-sensitive and/or pH-sensitive.

In another embodiment of the stent grafts disclosed herein, the stent graft comprises polymeric micelles incorporated within a coating applied to the stent graft wherein the coating is formulated to be a quick-release coating, a medium-release coating or a slow-release coating.

In another embodiment of the stent grafts disclosed herein, the delivery device is a pouch associated with the stent graft.

In another embodiment of the stent grafts disclosed herein, the pouch is created by providing a stent graft with two layers and partially adhering the layers together to form one or more pouches wherein following deployment the first layer is exposed to blood flow and the second layer faces the blood vessel wall and is semi-permeable.

Aneurysm treatment kits are also disclosed herein. One embodiment of an aneurysm treatment kit disclosed herein includes an aneurysm treatment kit comprising: a stent graft; a stent graft delivery catheter; polymeric micelles containing at least one therapeutic agent; and an energy source.

In another embodiment of the treatment kits disclosed herein, the energy source is selected from the group consisting of an ultrasonic energy source, a radiofrequency (RF) energy sources, an ultraviolet (UV) energy source, an infrared (IR) energy source, and a visible light source.

### BRIEF DESCRIPTION OF THE FIGURES

FIG. 1 depicts a fully deployed stent graft with an exterior metal scaffolding as used in an abdominal aortic aneurysm;

FIG. 2 depicts a delivery device associated with a stent graft deployed at an aneurysm site;

FIG. 3a is a side view of a pouch delivery device;

FIG. 3b is a cross-sectional view of a stent graft with a pouch delivery device wrapped around its outer surface;

FIG. 4 depicts a stent graft delivery catheter adapted to allow coating of the outer wall of a stent graft with polymeric micelles within the delivery catheter;

FIG. 5 depicts an alternative stent graft delivery catheter adapted to allow coating of the outer wall of a stent graft with polymeric micelles within the delivery catheter;

FIGS. 6a-6c depict stent graft deployment with the delivery of polymeric micelles through an injection catheter at the treatment site;

FIGS. 7a-c depict stent graft deployment with the delivery of polymeric micelles through injection catheters at the treatment site;

FIG. 8 depicts an alternate method of delivering polymeric micelles directly into the aneurysm sac after deployment of a stent graft;

FIG. 9 depicts an alternate method of delivering polymeric micelles directly into the aneurysm sac after deployment of a stent graft; and

FIG. 10 depicts yet another alternate method of delivering polymeric micelles directly into the aneurysm sac after deployment of a stent graft.

### DETAILED DESCRIPTION

An aneurysm is a swelling, or expansion of a blood vessel and is generally associated with a vessel wall defect. Previous methods to treat aneurysms involved highly invasive surgical procedures where the affected vessel region was removed (or opened) and replaced (or supplemented internally) with a synthetic graft that was sutured in place. However, this procedure is highly invasive and not appropriate for all patients. Historically, patients who were not candidates for this procedure remained untreated and thus at continued risk for sudden death due to aneurysm rupture.

To overcome some of the risks associated with invasive aneurysmal surgeries, stent grafts were developed. Stent grafts can be positioned and deployed using minimally invasive procedures. Essentially, a catheter having a stent graft compressed and fitted into the catheter's distal tip is advanced through an artery to a position spanning the aneurysmal site. The stent graft is then deployed within the vessel lumen juxtaposed to the weakened vessel wall forming an inner liner that insulates the aneurysm from passing blood flow and its resulting hemodynamic forces that can promote stress and rupture. The size and shape of the stent graft is matched to the treatment site's lumen diameter and aneurysm length.

Stent grafts generally comprise a metal scaffolding having a biocompatible graft material lining or covering such as Dacron®, ePTFE, or a fabric-like material woven from a variety of biocompatible polymer fibers. The graft material can be stitched, glued or molded to the scaffold. When a self-expanding stent graft is deployed from the delivery catheter, the scaffolding expands the graft material to fill the lumen and exerts radial force against the lumen wall.

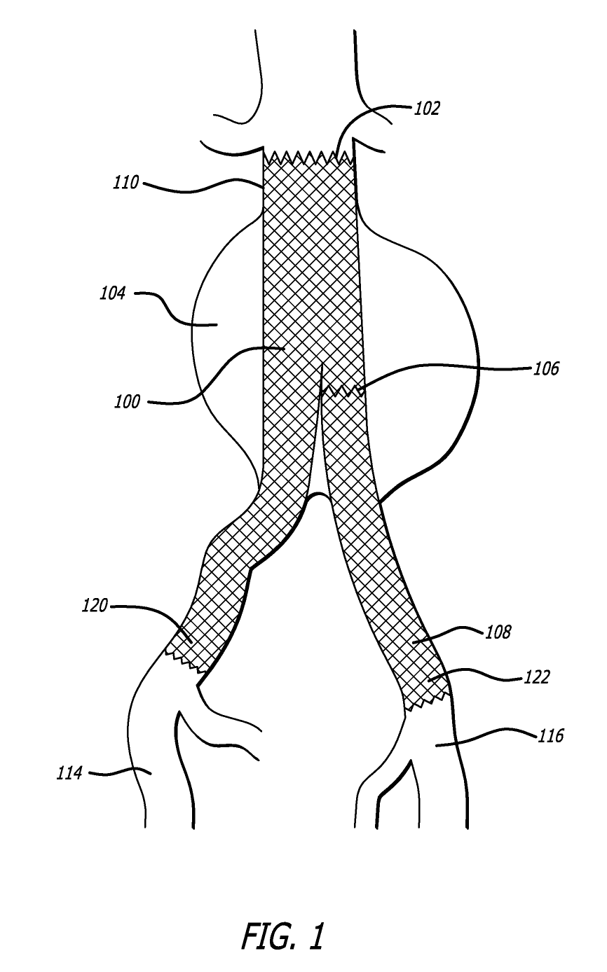

FIG. 1 depicts an exemplary stent graft placement at the site of an abdominal aortic aneurysm. In this type of placement, a stent graft 100 is deployed through the left iliac artery 114 to an aneurysm sac (site) 104. Stent graft 100 has a distal end 102 and an iliac leg 108 to anchor the stent graft in the right iliac artery 116. Stent graft 100 is deployed first in a first deployment catheter and iliac leg (limb) 108 is deployed in a second deployment catheter and the two segments are joined at overlap 106. Furthermore, after deployment, stent graft 100 contacts the blood vessel wall at least at sites 110, 120 and 122 to prevent leakage of blood into the aneurysm sac at these points.

While stent grafting such as that depicted in FIG. 1 can reduce the possibility of aneurysm rupture, it does not treat the aneurysm itself. That is, even though bypassed and insulated, the aneurysm and its associated diseased tissue remains. The aneurysmal tissue then can continue to degenerate such that the aneurysm continues to increase in size due to the continued thinning of the vessel wall. Thus, methods to treat the diseased tissue in addition to (or in place of) stent grafting would provide a significant advancement in the treatment of aneurysms. Moreover, it would be desirable to provide treatments to the aneurysm that can be controlled even following placement at the aneurysm site.

In general, the methods disclosed herein involve delivering polymeric micelles, which are formed of polymeric materials and one or more therapeutic agents, at an aneurysm site and exposing the polymeric micelles to energy sufficient to release or affect the rate of release of the therapeutic agent(s). As used herein, a “sufficient” amount of energy is the minimum amount of energy necessary to cause the release of or to increase the rate of release of a therapeutic agent from the polymeric micelles disclosed herein. Energy higher than that which is “sufficient” can also be used and is within the definition of sufficient as provided and claimed herein.

The polymeric micelles generally will be self-assembled block copolymers that consist of hydrophobic and hydrophilic regions. They will generally be spherical and have diameters ranging from about 10 microns to about 3000 microns. Polymeric micelles are also generally formed of a porous polymeric material.

Therapeutic agent(s) can escape from the polymeric material when the polymeric micelles are exposed to energy, in one example, ultrasound energy. Without being bound by theory, it is believed that upon exposing polymeric micelles to ultrasound, for example, the thermal energy produced by the ultrasound can increase the size of the pores in which the therapeutic agent(s) are stored to release or increase the rate of release of at least some of the therapeutic agent(s) from the polymeric micelles. It is also believed that vibrational energy imparted to polymeric micelles from the ultrasound can release or increase the release rate of the therapeutic agent(s) from the polymeric micelles.

The rate of release of the therapeutic agent(s) from the polymeric micelles can depend on the size of the pores within the polymeric structure of the polymeric micelles. The rate of release typically increases as the pores increase in size and typically decreases as the pores decrease in size. In certain embodiments, the polymeric micelles are formed from one or more macroporous polymers. Macroporous polymers typically have a pore size in the range of about 500 angstrom to about 1.0 micron (e.g., about 500 angstrom to about 0.75 micron, about 500 angstrom to about 0.5 micron, about 500 angstrom to about 0.25 micron, about 750 angstrom to about 0.75 micron, 0.1 micron to about 0.5 micron).

Alternatively or additionally, polymeric micelles can be formed from one or more microporous polymers. Microporous polymers typically have a pore size of about 100 angstrom to about 500 angstrom (e.g., about 200 angstrom to about 500 angstrom, about 300 angstrom to about 500 angstrom, about 400 angstrom to about 500 angstrom, about 300 angstrom to about 400 angstrom). The microporous polymer or polymers from which polymeric micelles are formed, for example, can be loaded with macro molecular therapeutic agent(s). See, for example, Rhine et al., J. of Pharmaceutical Sci., 69: 265-263 (1980) which is incorporated by reference in its entirety herein.

Polymeric micelles can be formed using any of various systems and techniques, such as emulsion polymerization and/or droplet polymerization techniques. Examples of droplet polymerization systems and techniques are described in, for example, US Patent Publication No. US 2003/0185896, published Oct. 2, 2003 and in US Patent Application No. US 2004/0096662, published May 20, 2004, each of which is incorporated by reference in its entirety herein.

Exposure of polymeric micelles to ultrasound energy, as noted above, can release or increase the rate of release of the therapeutic agent(s) from the polymeric micelles. In one example, an ultrasonic device can be positioned external to the subject (e.g., above skin) and activated. The ultrasound, for example, can be transmitted from the ultrasonic device through the skin, through tissue and to the aneurysm site where it reaches the polymeric micelles, causing the release of the therapeutic agent(s).

The intensity of the ultrasound transmitted from the ultrasonic energy device to the aneurysm treatment site can be a function of the distance between the skin and the aneurysm treatment site. For example, as the distance between skin and the aneurysm treatment site increases, the intensity of the energy used to release the therapeutic agent(s) can increase. Likewise, as the distance between skin and the aneurysm treatment site decreases, the intensity of the energy used to release the therapeutic agent(s) can decrease.

The release and/or rate of release of the therapeutic agent(s) from the polymeric micelles can be regulated by varying one or more of the frequency, duration, and/or intensity of the ultrasound energy. For example, increasing the frequency, duration, and/or intensity of the ultrasound energy can increase the likelihood and/or the rate of release of the therapeutic agent(s) from the polymeric micelles. Similarly, decreasing the frequency, duration, and/or intensity of the ultrasound energy can decrease the likelihood and/or the rate of release of the therapeutic agent(s) from the polymeric micelles. The frequency, duration, and/or intensity can be increased or decreased depending on various factors, such as the depth of the aneurysm treatment site beneath the skin and the targeted dosage of the therapeutic agent to be released. For example, it may be beneficial to increase the frequency, duration, and/or intensity as the depth of the aneurysm treatment site beneath the skin increases and/or the targeted dosage increases. Similarly, it may be beneficial to decrease the frequency, duration, and/or intensity as the depth of aneurysm treatment site beneath the skin and/or the targeted dosage decreases.

The frequency of the ultrasound energy transmitted to the polymeric micelles typically can range from about 20 kHz to about 10 MHz (e.g., from about 20 kHz to about 500 kHz; from about 50 kHz to about 200 kHz; from about 100 kHz to about 800 kHz; from about 800 kHz to about 2 MHz; from about 50 kHz to about 5 MHz; from about 1 MHz to about 8 MHz; from about 2 MHz to about 6 MHz; from about 1 MHz to about 10 MHz; from about 60 kHz to about 400 kHz; from about 200 kHz to about 250 kHz; from about 490 kHz to about 900 kHz). Each transmission of the ultrasound energy can last, for example, about ten seconds or longer (e.g, about 20 seconds or longer, about 30 seconds or longer, about 45 seconds or longer, about one minute or longer, about two minutes or longer, about four minutes or longer, about six minutes or longer, about eight minutes or longer, about ten minutes or longer, from about 20 seconds to about ten minutes, from about 20 seconds to about five minutes, from about 20 seconds to about one minute). The intensity of the ultrasound energy can range, for example, from about 0.1 W/cm² to about 30 W/cm² (e.g., from about one W/cm² to about 50 W/cm²). The ultrasound energy can be transmitted to the polymeric micelles in a continuous fashion or in a pulsed fashion.

In some embodiments, the ultrasound energy can be transmitted to the polymeric micelles in multiple intervals in order to release the therapeutic agent(s) in corresponding intervals. Polymeric micelles, for example, can include a sufficient amount of the therapeutic agent(s) to allow the release of the therapeutic agent(s) in response to each of multiple transmissions of ultrasound energy. As a result, the subject can receive multiple treatments with only one injection of the polymeric micelles. In some embodiments, the therapeutic agent(s) can be released from the polymeric micelles at least one time (e.g., at least about two times, at least about four times, at least about six times, at least about eight times, at least about ten times, at least about 20 times, at least about 30 times, etc.) before the ultrasound is rendered incapable of releasing anymore therapeutic agent(s) from the polymeric micelles (e.g., before the therapeutic agent(s) is/are completely expended from polymeric micelles).

The number and frequency of intervals in which the ultrasound is transmitted to the polymeric micelles can depend on a variety factors, such as the severity or size of the aneurysm and/or therapeutic agent(s) being used. The ultrasound energy, for example, can be transmitted to the polymeric micelles at least about one time per month (e.g., at least about three times per month, at least about five times per month, at least about ten times per month, at least about 20 times per month, at least about one time per week, at least about three times per week, at least about five times per week, at least about one time per day, at least about two times per day, at least about three times per day). The ultrasound energy can be transmitted for a predetermined time during the above-noted intervals. For example, the ultrasound energy can be transmitted for at least about ten seconds (e.g., at least about 20 seconds, at least about 30 seconds, at least about 45 seconds, at least about one minute, at least about five minutes, at least about ten minutes) during each of the intervals.

In some embodiments, the ultrasound is transmitted for the same or a similar period of time, at the same or a similar intensity, and/or at the same or a similar frequency for each interval. In certain embodiments, however, the duration, intensity, and/or frequency of energy transmission can increase or decrease as the treatment progresses. For example, it may be beneficial, in some cases, to decrease the duration, intensity, and/or frequency near the end of a treatment cycle (e.g., after a predetermined number of intervals).

Similarly, as the treatment progresses, the subject's need for the therapeutic agent(s) may decrease making it beneficial to decrease the duration, intensity, and/or frequency of treatments. For certain treatments, it might be beneficial to ramp up the dosage of therapeutic agent(s) released. In certain embodiments, the duration, intensity, and/or frequency can be increased after a predetermined number of treatments. In some cases, the duration, intensity, and/or frequency can gradually increase or decrease with each interval.

As an alternative to, or in addition to, ultrasound energy, for example, any other of various forms of energy can be used to release or increase the rate of release of the therapeutic agent(s) from the polymeric micelles including, without limitation, radiofrequency (RF) energy, ultraviolet (UV) energy, infrared (IR) energy, and/or visible light. Sources of each described energy type are well known to those of ordinary skill in the art with numerous available commercial sources.

While, in the embodiments discussed above, polymeric micelles are formed of a porous polymeric material, polymeric micelles can be formed of any of various other polymeric structures. In some embodiments, polymeric micelles are formed of nonporous polymers, such as hydrogels. Hydrogels can have internal structure based on molecular chains of entangled, cross-linked, and/or crystalline chain networks in the polymer. The therapeutic agent(s) can be contained within a space between the molecular chains. The space between macromolecular chains of hydrogels is referred to as the mesh size. Examples of hydrogels include polyhydroxyethylmethacrylate, polyvinyl alcohol, polyanhydrides, polyglycolides, and polylactides.

In certain embodiments, polymeric micelles are formed of cross-linked polymer chains, which can produce a screening effect to releasably retain the therapeutic agent(s) within the polymeric micelles. In some embodiments, when polymeric micelles formed of cross-linked polymer chains are exposed to certain types of energy, such as ultrasound and/or RF energy, the energy can cause the cross-linked structure to degrade resulting in the release of the therapeutic agent(s) from the polymeric micelles. For example, the cross-linked polymer can include bonds that break upon exposure to localized elevated temperatures produced by the energy. Examples of such bonds include ester or acids with amine introduced into the polymer by side chain reactions. In some embodiments, the vibrations produced by certain types of energy, such as ultrasound, can break bonds within the polymeric structure. The vibrations, for example, can cause one or more of the polymer chains to become cleaved. Consequently, the therapeutic agent(s) can be released from polymeric micelles. Examples of polymeric materials suitable for use in this embodiment include, but are not limited to, poly(L-lysine-co-polyethyleneglycol), poly(methacrylic acid-co-methacryloxyethylglucoside) and poly(methacrylic acid-co-ethyleneglycol), polylactic acid (PLA), polyglycolic acid (PGA), polyamides, poly(ε-caprolactone), poly(orthoesters), and polyanhydrides. Further non-limiting examples of suitable polymers in forming the coating include polyanhydrides, ethylene-vinyl acetate, poly(lactic acid), poly(glutamic acid), poly(ε-caprolactone), lactic/glycolic acid copolymers, polyorthoesters, polyamides and the like. Any of various cross-linking agents can be used.

In certain embodiments, polymeric micelles are formed of entangled polymeric chains that can similarly be exposed to particular forms of energy to create a localized temperature increase and/or vibrations that can cause the release or increase the rate of release of the therapeutic agent(s) from polymeric micelles. The heat and/or vibrations caused by ultrasound and/or RF energy, for example, can increase the mesh size of the entangled polymeric structure to release or increase the rate of release of the therapeutic agent(s) from polymeric micelles.

In some embodiments, polymeric micelles can be formed of one or more polymeric materials including a pendant group that can be solubilized. Solubilization of water-insoluble polymers, for example, can occur as a result of hydrolysis, ionization, or protonation of a side group. When polymeric micelles formed of such materials are exposed to certain types of energy, such as ultrasound, the energy can cause the release or increase the rate of release of the therapeutic agent(s) from the polymeric micelle. Polymers of this type include, for example, poly(L-lysine-co-polyethyleneglycol), poly(methacrylic acid-co-methacryloxyethylglucoside), and poly(methacrylic acid-co-ethyleneglycol).

In some embodiments, polymeric micelles are formed of a polymeric structure including a reservoir system in which a polymeric membrane surrounds a core of therapeutic agent(s). In this embodiment, a porous or non-porous polymer encapsulates therapeutic agent(s) within micro- or nano-polymeric micelles, which form micro-containers or micelles for the therapeutic agent(s). Non-limiting examples of polymers that can be used in this embodiment include poly(ethylene glycol) (PEG), poly(acrylic acid) (PAA) and poly(vinyl alcohol) (PVA) or co-polymers or block polymers thereof. See, for example, Tian and Uhrich, Polymer Preprints, 43(2): 719-720 (2002) which is incorporated by reference in its entirety herein. The polymer can be amphiphilic, containing controlled hydrophobic and hydrophilic balance (HLB), which can facilitate organization of the polymer into circular micelles. When polymeric micelles including such a reservoir system are exposed to certain types of energy, such as ultrasound and/or RF energy, the energy can alter the polymeric structure to release at least some of the therapeutic agent(s) from the polymeric micelles. Examples of suitable polymers with which reservoir systems can be formed include hydrogels such as swollen poly(2-hydroxyethyl methacrylate) (PEMA), silicone networks, and ethylene vinyl acetate copolymers. Further examples include polyvinyl alcohol, polyvinyl pyrrolidone, and polyethylene oxide. Other polymers can also be used. See, for example, Pedley et al., Br. Polymer J., 12: 99 (1980) which is incorporated by reference in its entirety herein.

In certain embodiments, the polymeric material of the polymeric micelles includes micelles that surround the therapeutic agent(s). The micelles, for example, can include air bubbles. The micelles can have a diameter of about 0.01 micron to about 100 microns and a gas volume of about 5% to about 30% of the volume of the micelles. Application of ultrasound to polymeric micelles can cause the air bubbles in the micelles to pulsate. As a result, the air bubbles can become asymmetric at the air/liquid interface. The surface of such a pulsating asymmetric oscillation bubble can cause a steady eddying motion to be generated in the immediate adjoining liquid, often called microstreaming. This pulsating results in a localized shearing action that can be strong enough to cause fragmentation of the internal structures of the polymer. For example, main chain rupture may be induced by shock waves during cavitiation of the liquid medium.

In certain embodiments, polymeric micelles are formed of one or more polymers including a photosensitizer linked to the backbone or side chain of the backbone of the polymer. When exposed to a particular energy (e.g., light energy having a wavelength between about 200 nm and 800 nm), the polymer can release the therapeutic agent(s). In some embodiments, the therapeutic agent(s) may be linked via a side chain to the polymer backbone, and the photosensitizer may be linked to the same or different polymer backbone in the vicinity of the therapeutic agent(s). It is also possible to attach a photosensitizer directly to the therapeutic agent(s), or to interpose a photosensitizer between a linker and the therapeutic agent(s). Examples of polymers that can be used in these embodiments include co-polymers of N-(-2hydroxypropyl)methacrylamide and an enzymatically degradable oligopeptide poly (L-lysine-copolyethylene glycol).

In certain embodiments, polymeric micelles include a photoreactive compound or photosensitizer linked to a polymer backbone using an appropriate linker, which can release the therapeutic agent(s) upon being exposed to certain types of energy, such as UV energy, IR energy, and/or visible light. For example, photosensitizers can be bound to therapeutic agent(s) having aliphatic amino groups to form photoreactive/therapeutic agent complexes. Polymer backbones or co-polymer precursors, for example, may be derivatized to contain co-polymer side chains or “linkers” having active ester functionalities. The aliphatic amino groups of the complexes may be bound to the active ester functionalities of the linker by aminolysis reactions. These stable moieties may be formed into co-polymers to be used in the formation of polymeric micelles. Application of an appropriate form of energy, for example, can result in release of the therapeutic agent(s) from the polymer by breaking a bond to the linker. See, for example, N. L. Krinick et al., J. Biomater. Sci. Polymer Edn., 5(4): 303-324 (1994) which is incorporated by reference in its entirety herein. The photochemically reactive group can be furfuryl alcohol or mesochlorine6 monoethylene diamine disodium salt.

Photoreactive agents may be used in conjunction with one or more therapeutic agents linked to the polymeric material of polymeric micelles. The release of the therapeutic agents can be controlled, for example, by exposure of polymeric micelles to UV energy, IR energy, and/or visible light. Examples of polymers that can be used in this embodiment include copolymers of N(-2-hydroxypropyl)methacrylamide and a linker, such as poly(L-lysine-co-polyethylene glycol). Further, non-limiting examples of suitable polymers for this embodiment include poly(propylene glycol) (PPG), poly(vinyl alcohol) (PVA) and poly(acrylic acid) (PAA).

Photosensitizers useful for attachment to one or more therapeutic agents or linkers can include dabcyl succinimidyl ester, dabcyl sulfonyl chloride, malachite green isothiocyanate, QSY7 succinimidyl ester, SY9 succinimidyl ester, SY21 carboxylic acid succinimidyl ester, and/or SY35 acetic acid succinimidyl ester, which are commercially available from Invitrogen Life Sciences, Carlsbad, Calif. These photoreactive agents can absorb light in the range of from about 450 nm to about 650 nm.

In some embodiments, polymeric micelles include a polymeric material and one or more therapeutic agents joined by a linking moiety. The linking moiety can be attached at a first end to the polymeric material and at a second end via a photochemically reactive group to the therapeutic agent(s). See, for example, U.S. Pat. Nos. 5,263,992 and 6,179,817, which are incorporated by reference in their entirety herein. Exposure to UV energy, IR energy, and/or visible light, for example, can cause the photochemically reactive group to release the therapeutic agent(s).

In certain embodiments, therapeutic agents having, or derivatized to contain, reactive aliphatic amino groups can be bound to polymers having, or derivatized to contain, ester or acid functional groups. The ester or acid moieties, for example, may be present on a polymer or co-polymer side chain. Amidization reaction can bind the aliphatic amino groups of the therapeutic agent to the ester groups on the polymer.

In certain embodiments, polymeric micelles include a linker having a photoreactive group arranged between a polymeric material and a therapeutic agent. The photoreactive group and the therapeutic agent may be embedded in the polymeric material or coated on a surface thereof. The photoreactive group, for example, can release the therapeutic agent upon exposure to light in the wavelength range of from about 200 nm to about 800 nm.

In some embodiments, the polymeric material used to form the polymeric micelles includes both bonds and pores that react upon exposure to ultrasound energy so as to release the therapeutic agent(s).

In certain embodiments, polymeric micelles can be coated with one or more of the polymers or polymer systems discussed above, which can contain one or more therapeutic agents. Polymeric micelles, for example, can include a polyvinyl alcohol matrix polymer surrounded by a sodium alginate coating that contains the therapeutic agent(s). The polymeric material of the coating can be adapted to controllably release the therapeutic agents upon exposure to one or more forms of energy. Polymeric micelles having coatings are disclosed, for example, in US Patent Publication No. 2004/0076582 A1, published on Apr. 22, 2004, which is incorporated by reference in its entirety herein.

While some examples of polymeric materials from which polymeric micelles can be formed have been described, one or more of the polymeric materials listed below can alternatively or additionally be used. For example, polymeric micelles can be formed of poly(glycolic acid) (PGA), poly(L-lactic acid) (PLLA) (PLA), polyoxalates, poly(α-esters), polyanhydrides, polyacetates, polycaprolactones, poly(orthoesters), polyamino acids, polyurethanes, polycarbonates, polyiminocarbonates, polyamides, poly(alky cyanoacrylates), and mixtures and copolymers thereof. Additional examples of polymeric materials include, stereopolymers of L- and D-lactic acid, copolymers of 1,3bis(p-carboxyphenoxy)propane and sebacic acid, sebacic acid copolymers, copolymers of caprolactone, poly(lactic acid)/poly(glycolic acid)/polethyleneglycol terpolymers, copolymers of polyurethane and poly(lactic acid), copolymers of α-amino acids, copolymers of α-amino acids and caproic acid, copolymers of α-benzyl glutamate and polyethylene glycol, copolymers of poly succinic acid and poly(glycols), polyphosphazene, polyhdroxy-alkanoates and mixtures thereof.

In some embodiments, polymeric micelles are formed of poly(ethylene oxide) (PEO), poly(ethylene glycol) (PEG), poly(propylene glycol) (PPG), poly(L-lactic acid) (PLLA), poly(ε-caprolactone), poly(a-amino acids), polyurethanes, poly(vinyl alcohol) (PVA) poly(vinyl pyrrolidone), poly hydroethyl methacrylate, polyhydroxyethyl methacrylate, and/or copolymers thereof.

Examples of additional polymers from which polymeric micelles may be formed include poly (lactic acid-co-glycolic acid) (PLGA), poly (lactic acid-co-ε-caprolactone) (PLACL), PLA-PEG diblock copolymer, PLA-PEG-PLA triblock copolymer, poly(orthoesters), poly(sebactic anhydride), poly(acrylic acid) (PAA) and derivatives, poly(ethylene-co-vinylacetate) (PEVAc), poly(lysine), poly(lactic acid-co-lysine), polyurethanes and block copolymers (e.g., commercially available polyurethanes such as, without limitation, BIOMER, ACUTHANE (available from Dow Chemical Co.) and PELLETHANE (available from Dow Chemical Co.) and poly(dimethylsiloxanes).

Further examples of commercially available polymers that can be used to form polymeric micelles include PLURONIC (available from BASF Corp.); MEDISORB, ELVAX40P (ethylene vinyl acetate) and BIODEL (available from Dupont Corp.); Polymer No. 6529C (Poly(lactic acid)) and Polymer No. 6525 (poly(glycolic acid)) (both available from Polysciences Inc.).

The size of polymeric micelles need not be limited to the sizes discussed above. In certain embodiments, polymeric micelles have a diameter of no greater than about 10,000 microns (e.g., no greater than about 7,500 microns, no greater than about 5,000 microns, no greater than about 2,500 microns, no greater than about 2,000 microns, no greater than about 1,5000 microns, no greater than about 1,000 microns, no greater than about 500 microns, no greater than about 400 microns, no greater than about 300 microns, no greater than about 200 microns, no greater than about 100 microns). In some embodiments, polymeric micelles have a diameter of about 100 microns to about 10,000 microns (e.g., about 100 microns to about 1000 microns, about 100 microns to 500 microns, about 2,500 microns to about 5,000 microns, about 5,000 microns to about 10,000 microns, about 7,500 microns to about 10,000 microns).

While the polymeric micelles described above are typically formed to be spherical, they can also be non-spherical. Non-spherical polymeric micelles, for example, can be produced using techniques similar to those described above. Non-spherical polymeric micelles can be manufactured and formed, for example, by controlling drop formation conditions. In some embodiments, non-spherical polymeric micelles can be formed by post-processing the polymeric micelles (e.g., by cutting or dicing into other shapes). Polymeric micelle shaping is described, for example, in US Patent Publication No. US 2003/0203985 A1, published on Oct. 30, 2003 which is incorporated by reference in its entirety herein.

In certain embodiments, polymeric micelles include a core region and multiple layers surrounding the core region. One or more of the layers (e.g., coatings) can be, for example, a degradable and/or bioabsorbable polymer. The coating can be applied by dipping or spraying the polymeric micelles. The erodible polymer can be a polysaccharide (such as an alginate) or a polysaccharide derivative. In certain embodiments, the coating can be an inorganic, ionic salt. Other erodible coatings include water soluble polymers (such as a polyvinyl alcohol, e.g., that has not been cross-linked), biodegradable poly DL-lactide-poly ethylene glycol (PELA), hydrogels (e.g., polyacrylic acid, haluronic acid, gelatin, carboxymethyl cellulose), polyethylene glycols (PEG), chitosan, polyesters (e.g., polycaprolactones), and poly(lactic-co-glycolic) acids (e.g., poly(d-lactic-co-glycolic)acids).

In some embodiments, energy can be transmitted to polymeric micelle in multiple intervals to release the therapeutic agent(s) from the multiple layers of coating, respectively. After causing the release of the therapeutic agent(s) from the outermost layer, for example, the outermost layer can erode. As a result, the next transmission of energy can cause the release of the therapeutic agent(s) from the next outermost layer without impedance of the outermost layer that has eroded. In certain embodiments, some of the multiple layers can include different therapeutic agents such that sequential exposures to energy can be used to release different therapeutic agents.

In some embodiments, polymeric micelles can include a core that includes one or more therapeutic agents and a coating that includes one or more therapeutic agents. The therapeutic agent(s) in the coating can be the same as or different than the therapeutic agent(s) in the core. Energy can be transmitted to polymeric micelles to release the therapeutic agents of the core and coating simultaneously or sequentially. Examples of polymeric micelles having one or more therapeutic agents in a core and in one or more layers surrounding the core (e.g., one ore more coatings) can be found, for example, in US Patent Publication No. US 2004/0076582 A1, published on Apr. 22, 2004, which is incorporated by reference in its entirety herein.

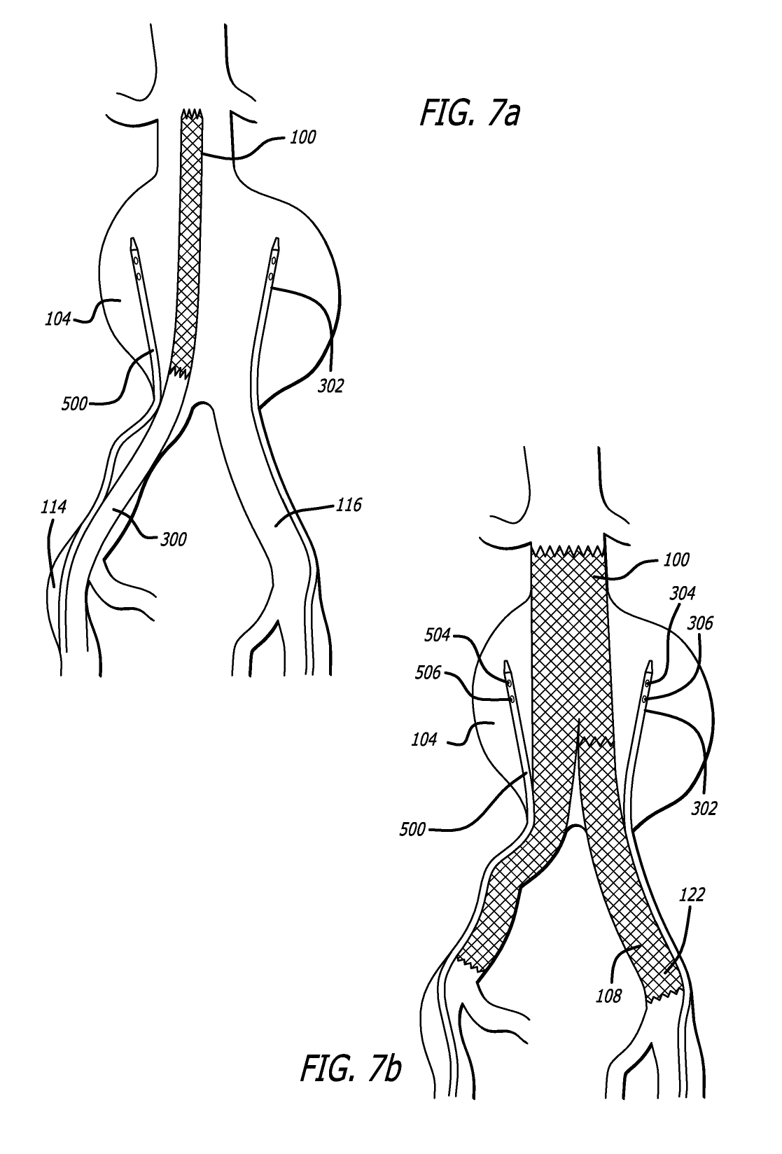

While the methods described above include the use of an extra-dermal energy device, other types of energy devices can be used. For example, energy can be transmitted to polymeric micelles by a local energy device deployed to the treatment site in a similar manner of the injection catheters as depicted in FIGS. 6-10. In this manner, a local energy device can be placed at the aneurysm site and activated. In certain embodiments, the energy device includes an energy emitting end. Energy device can then be activated to emit energy from the end. The energy can contact the polymeric micelles to release the therapeutic agent(s) contained therein. Use of a local energy device, for example, can allow energy to be transmitted to the polymeric micelles with substantially undiminished intensity. It may be beneficial to use a local energy device for transmitting particular forms of energy, such as UV energy, IR energy, and visible light, that may be less able to penetrate multiple layers of tissue.

In certain embodiments, some of the polymeric micelles include a therapeutic agent that can be released when exposed to a first type of energy, such as, for example, ultrasound, and some of the polymeric micelles include a therapeutic agent that can be released when exposed to a second type of energy, such as, for example, visible light. In some embodiments, some of the polymeric micelles include a therapeutic agent that can be released when exposed to ultrasound, and some of the polymeric micelles include a therapeutic agent that can be released when exposed to UV energy. In some embodiments, some of the polymeric micelles include a therapeutic agent that can be released when exposed to ultrasound, and some of the polymeric micelles include a therapeutic agent that can be released when exposed to IR energy. In some embodiments, some of the polymeric micelles include a therapeutic agent that can be released when exposed to ultrasound, and some of the polymeric micelles include a therapeutic agent that can be released when exposed to RF energy. In some embodiments, some of the polymeric micelles include a therapeutic agent that can be released when exposed to RF energy, and some of the polymeric micelles include a therapeutic agent that can be released when exposed to visible light. In some embodiments, some of the polymeric micelles include a therapeutic agent that can be released when exposed to RF energy, and some of the polymeric micelles include a therapeutic agent that can be released when exposed to UV energy. In some embodiments, some of the polymeric micelles include a therapeutic agent that can be released when exposed to RF energy, and some of the polymeric micelles include a therapeutic agent that can be released when exposed to IR energy. In some embodiments, some of the polymeric micelles include a therapeutic agent that can be released when exposed to visible light, and some of the polymeric micelles include a therapeutic agent that can be released when exposed to UV energy. In some embodiments, some of the polymeric micelles include a therapeutic agent that can be released when exposed to visible light, and some of the polymeric micelles include a therapeutic agent that can be released when exposed to IR energy. In some embodiments, some of the polymeric micelles include a therapeutic agent that can be released when exposed to IR energy, and some of the polymeric micelles include a therapeutic agent that can be released when exposed to UV energy.

Similarly, in some embodiments, some of the polymeric micelles include a therapeutic agent that can be released when exposed to a first intensity of energy, and some of the polymeric micelles include a therapeutic agent that can be released when exposed to a second intensity of the same form of energy.

In certain embodiments, one or more of the polymeric micelles can include a super-absorbable polymer and/or a shape-memory material (e.g., a polymer). Examples of super-absorbable polymers include Merocel® polymer. Examples of shape-memory polymer materials are known in the art. Shape memory materials and polymeric micelles that include shape memory materials are described in, for example, US Patent Publication No. 2004/0091543 published May 13, 2004 and US Patent Publication No. 2005/0095428 published May 5, 2005 both of which are incorporated by reference in their entirety herein.

Now that the polymeric micelles have been described, methods of delivering the polymeric micelles to the aneurysm site are described.

1. Placement Onto Stent Graft and/or Stent Graft Coatings

Polymeric micelles(s) can be applied to the surface of a stent graft. Following stent graft deployment, the polymeric micelle(s) will diffuse off of the stent graft material to the aneurysm treatment site. When this embodiment is used, polymeric micelle(s) can be applied to the surface of the stent graft using methods including, but not limited to, precipitation, coacervation or crystallization. The polymeric micelle(s) can also be bound to the stent graft covalently, ionically, or through other intramolecular interactions including, without limitation, hydrogen bonding and van der Waals forces.

Polymeric micelle(s) can also be incorporated into a coating placed onto the stent graft. Thus, a stent graft coating is a material placed onto the fabric of a stent graft that can hold and release polymeric micelle(s).

Stent graft coatings used in accordance with the present disclosure can be either biodegradable or non-biodegradable. Non-limiting representative examples of materials that can be used to produce biodegradable coatings include, without limitation, albumin; collagen; gelatin; fibrinogen; hyaluronic acid; starch; cellulose and cellulose derivatives (e.g. methylcellulose, hydroxypropylcellulose, hydroxypropylmethylcellulose, carboxymethylcellulose, cellulose acetate phthalate, cellulose acetate succinate, hydroxypropylmethylcellulose phthalate); casein; dextran; polysaccharides; poly(lactic acid); poly(D,L-lactide); poly(D,L-lactide-co-glycolide); poly(glycolide); poly(hydroxybutyrate); poly(alkylcarbonate); polyesters; poly(orthoesters); poly(ester amide)s (e.g. based on 1,4-butanediol, adipic acid, and 1,6-aminohexanoic acid (BAK 1095)); poly(ester carbonate)s (e.g. tyrosine-poly(alkylene oxide)-derived poly(ether carbonate)s; poly(hydroxyvaleric acid); polydioxanone; poly(malic acid); poly(tartronic acid); polyanhydrides (e.g. poly(adipic anhydride) and poly(sebacic acid-co-1,3-bis(p-carboxyphenoxy)propane)); polyphosphazenes; poly(amino acids); poly(trimethylene carbonate); poly(hydroxy valerate); poly(hydroxy butyrate-co-hydroxy valerate); poly(butylene succinate) (e.g. Bionolle®); poly(butylene adipate); polyarylates (e.g. tyrosine-derived polyarylates); poly(butylene terephthalate)-poly(ethylene glycol) copolymers (polyActive®); poly(\acute{ε}-caprolactone)-b-poly(ethylene glycol)) block copolymers; and poly(ethylene oxide)-b-poly(hydroxy butyrate) block copolymers.

Non-limiting representative examples of materials that can be used to produce non-biodegradable coatings include poly(ethylene-vinyl acetate) (“EVA”) copolymers; silicone rubbers; acrylic polymers (e.g. polyacrylic acid, polymethylacrylic acid, polymethylmethacrylate, polyalkylcynoacrylate); polyethylene; polypropylene; polyamides (nylon 6,6); polyurethane; poly(ester urethanes); poly(ether urethanes); poly(ester-urea); polyethers (e.g. based on poly(oxyethylene) and poly(oxypropylene) units (Pluronic®)); poly(ethylene oxide); poly(propylene oxide); other pluronics; poly(tetramethylene glycol)); and vinyl polymers (e.g. polyvinylpyrrolidone, poly(vinyl alcohol), poly(vinyl acetate phthalate and poly(vinylchloride).

Other useful materials that can be used to produce coatings include polymers such as poly(D,L-lactic acid); poly(L-lactic acid); poly(glycolic acid); poly(caprolactone); poly(valerolactone); copolymers of poly(caprolactone) or poly(lactic acid) with a polyethylene glycol (e.g., MePEG); carboxylic polymers; polyacetates; polyacrylamides; polycarbonates; polyvinylbutyrals; polysilanes; polyureas; polyoxides; polystyrenes; polysulfides; polysulfones; polysulfonides; polyvinylhalides; pyrrolidones; cross-linkable acrylic and methacrylic polymers; vinyl acetate polymers; vinyl acetal polymers; epoxy; melamine; phenolic polymers; water-insoluble cellulose ester polymers (e.g. cellulose acetate propionate, cellulose acetate, cellulose acetate butyrate, cellulose nitrate, and mixtures thereof); polyethylene oxide; polyhydroxyacrylate; poly(ethylene terephthalate); xanthan; hydroxypropyl cellulose; vinyllactam; vinyl butyrolactam; vinyl caprolactam; other vinyl compounds having polar pendant groups; acrylate and methacrylate having hydrophilic esterifying groups; hydroxyacrylate; cellulose esters and ethers; ethyl cellulose; hydroxyethyl cellulose; polyacrylate; natural and synthetic elastomers; rubber; acetal; nylon; styrene polybutadiene; acrylic resin; polycarbonate; polyvinylchloride; polyvinylchloride acetate; pectin; sucrose acetate isobutyrate; hydroxyapatite; tricalcium phosphate; silicates (e.g. Bioglass®, montmorillonite, and mica); alginate; poly(acrylic acid); poly-L-lysine; polyethylenimine; poly(allyl amine); fluorinated polyolefins (e.g. polytetrafluorethylene (Teflon®)); poly(N-isopropylacrylamide); polyacetals; aromatic polyesters; poly(propylene terephthalate) (Sorona®); poly(ether ether ketone)s; and poly(ester imide)s. In general, see U.S. Pat. No. 6,514,515 to Williams; U.S. Pat. No. 6,506,410 to Park, et al.; U.S. Pat. No. 6,531,154 to Mathiowitz, et al.; U.S. Pat. No. 6,344,035 to Chudzik, et al.; U.S. Pat. No. 6,376,742 to Zdrahala, et al.; Griffith, L. A., Ann. N.Y. Acad. of Sciences, 961:83-95 (2002); and Chaikof, et al, Ann. N.Y. Acad. of Sciences, 961:96-105 (2002) the entire contents of each of which are incorporated by reference herein. Additionally, all materials described herein can be blended or copolymerized in various compositions as appropriate, beneficial or required. Such blending or copolymerization is within the level of the ordinary skill in the art.

The selected material used in a particular coating can be obtained from various chemical companies known to those of ordinary skill in the art. However, when polymers are selected as a coating material, because of the potential presence of unreacted monomers, low molecular weight oligomers, catalysts, or other impurities in such commercially available polymers, it can be desirable (or, depending upon the materials used, necessary) to increase the purity of the selected polymer. Such a purification process yields polymers of better-known, purer composition, and therefore increases both the predictability and performance of the mechanical characteristics of the coatings. The exact purification process will depend on the polymer or polymers chosen. Generally, however, in a purification process, the polymer will be dissolved in a suitable solvent. Suitable solvents include (but are not limited to) methylene chloride, ethyl acetate, chloroform, and tetrahydrofuran. The polymer solution usually is then mixed with a second material that is miscible with the solvent, but in which the polymer is not soluble, so that the polymer (but not appreciable quantities of impurities or unreacted monomer) precipitates out of solution. For example, a methylene chloride solution of the polymer can be mixed with heptane, causing the polymer to fall out of solution. The solvent mixture then is removed from the copolymer precipitate using conventional techniques.

The coatings used in accordance with the present disclosure can be fashioned in a variety of forms with desired release characteristics and/or with other specific desired properties. For example, the coatings can be fashioned to release the polymeric micelle(s) upon exposure to a specific triggering event such as pH. Non-limiting representative examples of pH-sensitive coating materials include poly(acrylic acid) and its derivatives (e.g. homopolymers such as poly(aminocarboxylic acid); poly(acrylic acid); poly(methyl acrylic acid); copolymers of such homopolymers; and copolymers of poly(acrylic acid) and other acrylmonomers. Other pH sensitive polymers include polysaccharides such as cellulose acetate phthalate; hydroxypropylmethylcellulose phthalate; hydroxypropyl methylcellulose acetate succinate; cellulose acetate trimellilate; and chitosan. Yet other pH sensitive polymers include any mixture of a pH sensitive polymer and a water-soluble polymer.

Temperature-sensitive polymeric coatings can also be used. Non-limiting representative examples of temperature-sensitive materials and their gelatin temperature include homopolymers such as poly(N-methyl-N-n-propylacrylamide) (19.8° C.); poly(N-n-propylacrylamide) (21.5° C.); poly(N-methyl-N-isopropylacrylamide) (22.3° C.); poly(N-n-propylmethacrylamide (28.0° C.); poly(N-isopropylacrylamide) (30.9° C.); poly(N,n-diethylacrylamide) (32.0° C.); poly(N-isopropylmethacrylamide) (44.0° C.); poly(N-cyclopropylacrylamide) (45.5° C.); poly(N-ethylmethyacrylamide) (50.0° C.); poly(N-methyl-N-ethylacrylamide) (56.0° C.); poly(N-cyclopropylmethacrylamide) (59.0° C.); and poly(N-ethylacrylamide) (72.0° C.). Cellulose ether derivatives such as hydroxypropyl cellulose (41° C.); methyl cellulose (55° C.); hydroxypropylmethyl cellulose (66° C.); and ethylhydroxyethyl cellulose as well as pluronics such as F-127 (10-15° C.); L-122 (19° C.); L-92 (26° C.); L-81 (20° C.); and L-61 (24° C) can also be used. Moreover, temperature-sensitive materials can be made by preparing copolymers between (among) monomers of the above, or by combining such homopolymers with other water-soluble polymers such as acrylmonomers (e.g., acrylic acid and derivatives thereof such as methylacrylic acid, acrylate and derivatives thereof such as butyl methacrylate, acrylamide, and N-n-butyl acrylamide).

Polymers that release the micelles when exposed to energy as described above can also be used.

Coatings used in accordance with the present disclosure can be prepared in a variety of paste or gel forms. For example, within one embodiment, coatings are provided which are liquid at one temperature (e.g., a temperature greater than about 37° C., such as about 40° C., about 45° C., about 50° C., about 55° C. or about 60° C.), and solid or semi-solid at another temperature (e.g., ambient body temperature, or any temperature lower than about 37° C.). As is understood by one of ordinary skill in the art, such pastes or gels can be made utilizing a variety of techniques. Other pastes or gels can be applied as a liquid, which can solidify in vivo due to dissolution of a water-soluble component of the paste and precipitation of encapsulated therapeutic agent into the aqueous body environment.

Coatings can be fashioned in any appropriate thickness. For example, coatings can be less than about 2 mm thick, less than about 1 mm thick, less than about 0.75 mm thick, less than about 0.5 mm thick, less than about 0.25 mm thick, less than about 0.10 mm thick, less than about 50 μm thick, less than about 25 μm thick or less than about 10 μm thick. Generally, such coatings will be flexible with a good tensile strength (e.g., greater than about 50, greater than about 100, or greater than about 150 or 200 N/cm2), have good adhesive properties (i.e., adhere to moist or wet surfaces), and have controlled permeability.

As is understood by one of ordinary skill in the art, polymeric micelle(s) can be, without limitation, linked by occlusion in the matrices of a coating, bound by covalent linkages, or encapsulated in microcapsules within the coating.

Coatings used in accordance with the present disclosure can be formulated to deliver the polymeric micelle(s) over a period of about several minutes, several hours, several days, several months or several years. For example, “quick release” or “burst” coatings can release greater than about 10%; greater than about 20%, or greater than about 25% (w/v) of the polymeric micelle(s) over a period of about 7 to about 10 days. “Slow release” coatings can release less than about 1% (w/v) of the polymeric micelle(s) over a period of about 7 to about 10 days. “Medium-release” coatings can have release profiles between the quick-release and slow-release profiles.

In one embodiment, coatings can further be coated with a physical barrier to protect the coating during packaging, storage and deployment procedures. Physical barriers can also be used to affect the release profile of polymeric micelle(s) from the coating once the stent graft is deployed. Such barriers can include, without limitation, inert biodegradable materials such as gelatin, PLGA/MePEG film, PLA, or polyethylene glycol. In the case of PLGA/MePEG, once the PLGA/MePEG becomes exposed to blood, the MePEG will dissolve out of the PLGA, leaving channels through the PLGA to the underlying coating containing polymeric micelle(s).

Another example of a suitable physical barrier over the coating is an anti-coagulant (e.g. heparin), which can be applied over the top of the polymeric micelle(s)-containing coating. The presence of an anti-coagulant can delay coagulation. As the anticoagulant dissolves away, the anticoagulant activity stops, and the newly exposed polymeric micelle(s) coating can initiate their intended action.

In some embodiments, alternating layers of the polymeric micelle(s) coating with a protective coating can enhance the time-release properties of the coating overall.

The disclosed coatings can be applied according to any technique known to those of ordinary skill in the art of medical device manufacturing. For example, coatings can be applied to the stent grafts used as a “spray”, which solidifies into a coating. Such sprays can be prepared from microspheres of a wide array of sizes, including for example and without limitation, from about 0.1 μm to about 3 μm, from about 10 μm to about 30 μm or from about 30 μm to about 100 μm. Additionally or alternatively, coatings can be applied by, without limitation, impregnation, spraying, brushing, dipping and/or rolling. In another embodiment, a polymer-polymeric micelle(s) blend can be used to fabricate fibers or strands that are embedded within the fabric of the stent graft. After a coating is applied, it can be dried. Drying techniques include, but are not limited to, heated forced air, cooled forced air, vacuum drying or static evaporation.

For additional information regarding stents, stent grafts and coatings, see U.S. Pat. No. 6,387,121 to Alt; U.S. Pat. No. 6,451,373 to Hossainy, et al.; and U.S. Pat. No. 6,364,903 to Tseng, et al the entire contents of each of which are incorporated by reference herein.

2. Delivery Devices Associated with Stent Grafts

In place of, or in addition to, coatings on a stent graft, polymeric micelles can also be administered to an aneurysm site following stent graft deployment with the use of a delivery device associated with the stent graft. In these embodiments, the stent graft isolates the aneurysm site from blood flow and provides a structure to which the delivery device can be attached. In this manner, polymeric micelles can be delivered directly to the aneurysm site and not to surrounding healthy tissue. The polymeric micelles are released into this relatively sealed environment such that they are largely limited to this region. Thus, a maximum concentration of the polymeric micelles remains at the treatment site and are not delivered or distributed throughout the rest of the body. As a result, substantial quantities of the polymeric micelles remain at the treatment site for a longer period of time, increasing the efficacy of the polymeric micelles treatment.

Delivery devices can include, without limitation, a pouch that is attached to the stent graft or made from stent graft layers wherein the polymeric micelles (and associated carriers when used) are placed inside the pouch.

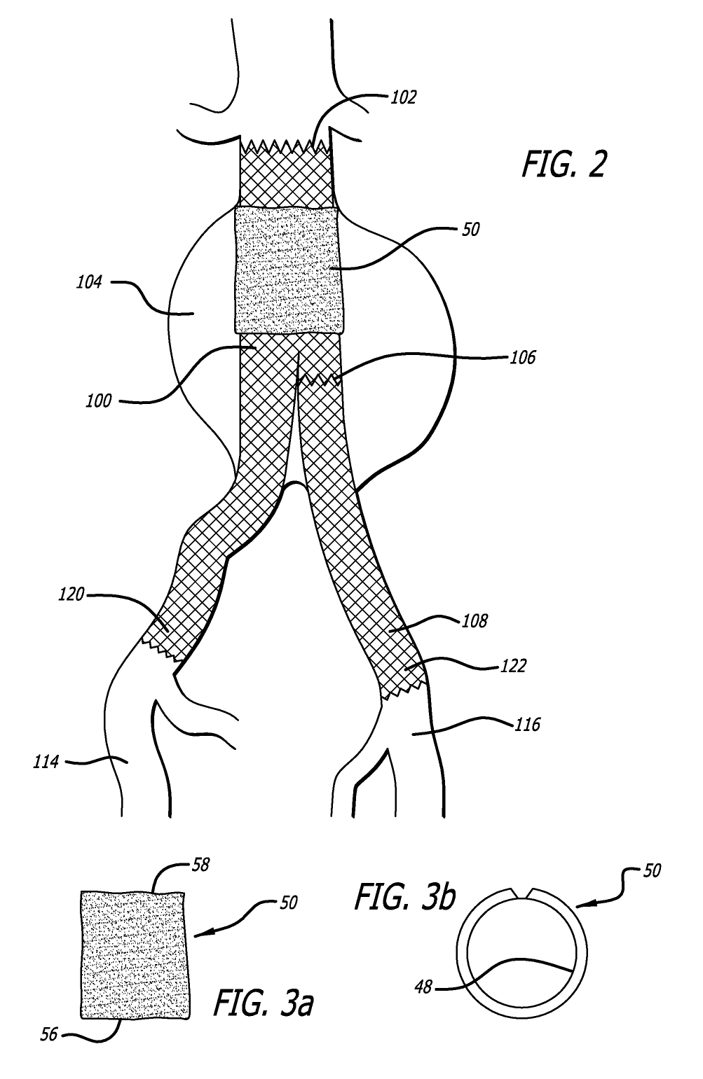

FIG. 2 depicts a polymeric micelles delivery device in the form of a pouch 50. In this depicted embodiment, the pouch 50 is connected to a ring 48 on the outer surface of the stent graft 22. The delivery device (50) is positioned such that upon placement at an aneurysm site (in the depicted example an aneurysmal sac 18 of aorta 10), the delivery device (50) is located between the stent graft 22 and the aneurysmal wall 16 of aorta 10.

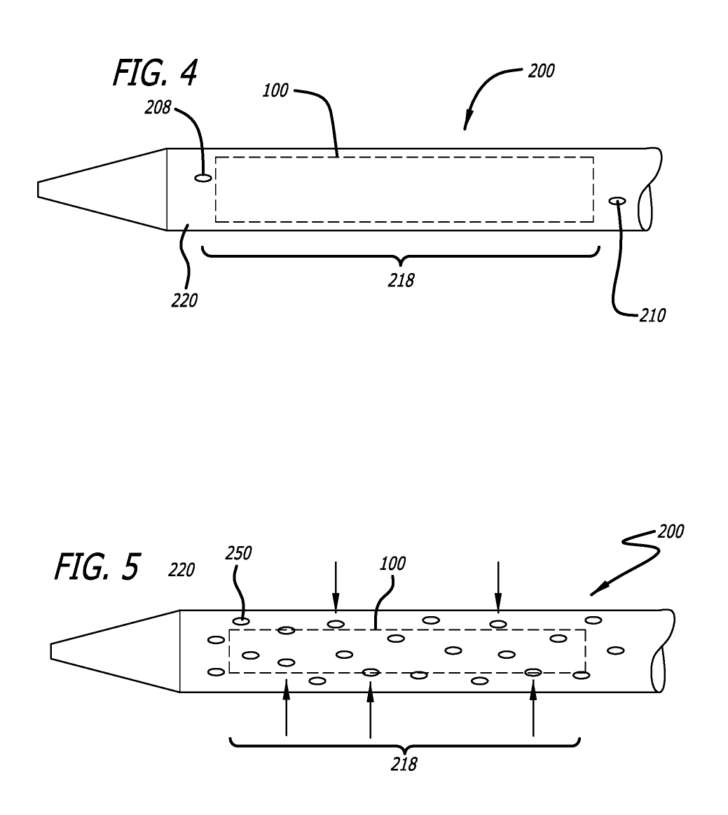

FIG. 3a depicts the pouch 50 delivery device. The pouch 50 can be wrapped around the outer wall of the stent graft and attached, in one embodiment, at end 58 of pouch 50. Pouch 50 can be prepared, for example, by folding a sheet of the pouch material in half, and attaching together the opposed sides projecting from the crease occurring at the fold which forms end 56, such as by sewing, laser welding, adhesives or the like to leave an open end. The polymeric micelles (with or without carriers) are then loaded into the interior of the pouch 50. The open end 58 can then be sealed. FIG. 3b shows a top cross-sectional view of pouch 50 attached to ring 48 of the stent graft 22.

Alternatively, multiple pouches can be used, with each pouch being attached to the stent graft. In one embodiment the pouches are arranged so that the spacing between adjacent pouches extending about the circumference of the stent graft is relatively equal. In one embodiment, at least four such delivery devices are equally spaced about the circumference of the stent graft. Alternatively, multiple delivery devices can be located both about the circumference of the stent graft, as well as longitudinally along the stent graft. In another embodiment, appropriately placed pouches can be created by adopting a stent graft that includes two fabric layers. The fabric layers can be adhered together at various places to create any desired number or configuration of pouches.

3. Delivery and/or Injection Catheters

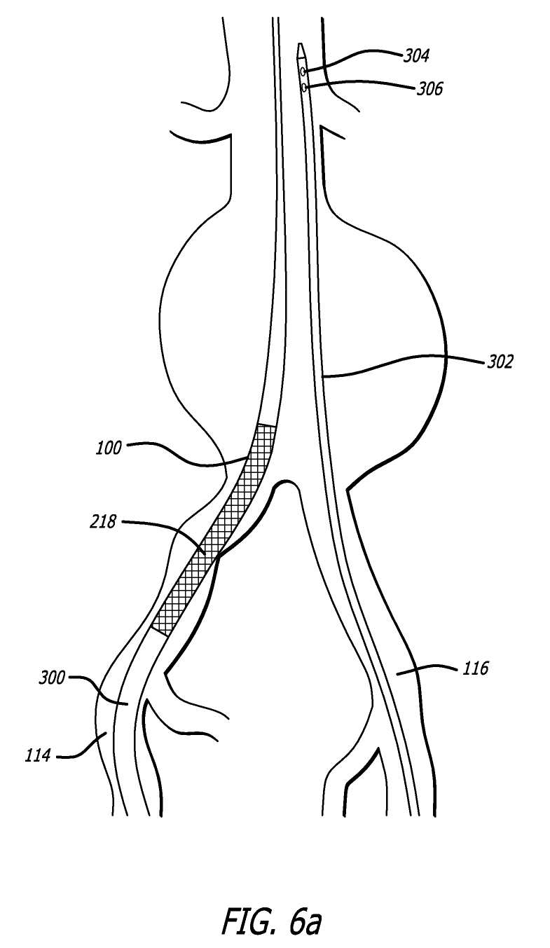

Polymeric micelles can also be delivered to the site of an aneurysm using delivery and/or injection catheters at or near the time of stent graft deployment. In one embodiment, a stent graft is pre-loaded into a delivery catheter such as that depicted in FIG. 4. Stent graft 100 is radially compressed to fill the stent graft chamber 218 in the distal end of delivery catheter 200. The stent graft 100 is covered with a retractable sheath 220. In this depicted embodiment, delivery catheter 200 has two injection ports 208 and 210 for applying polymeric micelles onto the outer wall of the stent graft prior to deployment. Stent graft 100 is then deployed to the treatment site as depicted in FIG. 1.

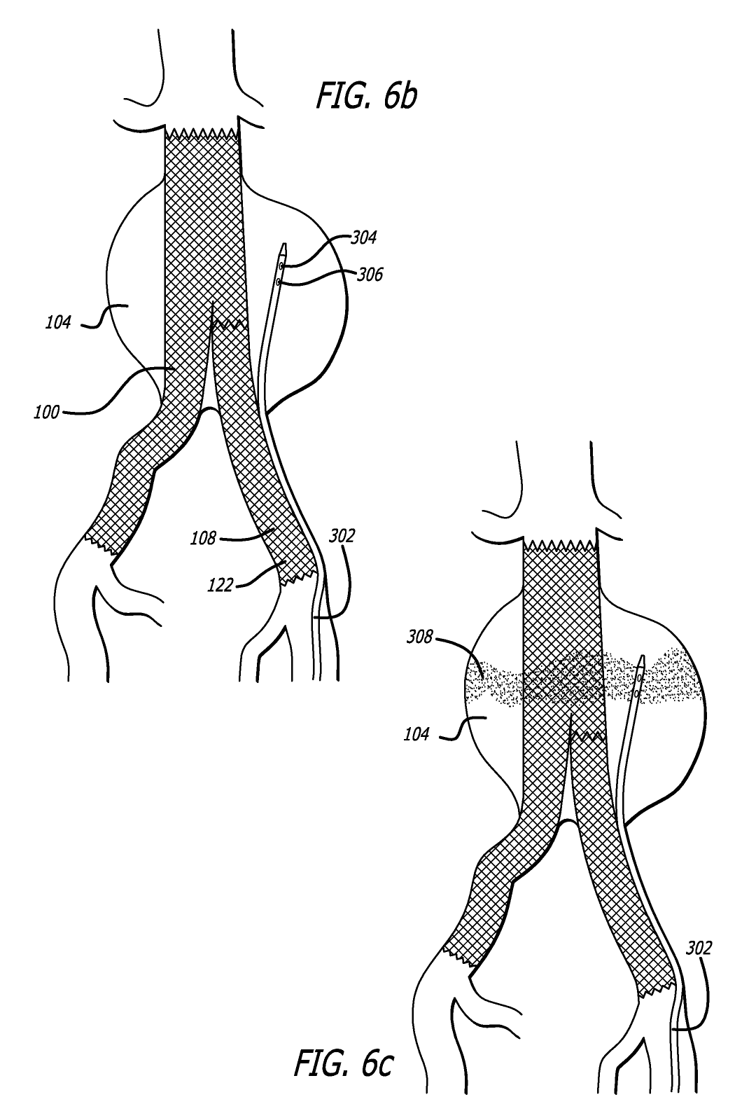

Another embodiment for coating the outer wall of the stent graft 100 within a delivery catheter 200 is depicted in FIG. 5. Retractable sheath 220 contains a plurality of holes 250 through which polymeric micelles can be applied to the outer wall of stent graft 100 compressed within stent graft chamber 218 prior to deployment. Stent graft 100 is then deployed to the treatment site as depicted in FIG. 1.

In another embodiment, polymeric micelles are injected between the stent graft and the vessel wall during or after stent graft placement. As depicted in FIG. 6a, a stent graft 100 is radially compressed to fill the stent graft chamber 218 of stent delivery catheter 300 which is then deployed to the treatment site via the left iliac artery 114. A multilumen injection catheter 302 is also deployed to the treatment site through the right iliac artery 116. The multilumen injection catheter 302 can be a coaxial catheter with two injection lumens or a dual lumen catheter or alternatively a three lumen catheter if a guide wire lumen is required. Injection catheter 302 has injection ports 304 and 306 through which a polymeric micelles can be delivered to a treatment site. In the first step of this deployment scheme (FIG. 6a), the stent delivery catheter 300 and the injection catheter 302 are deployed independently to the treatment site.

FIG. 6b shows stent graft 100 deployed. In this depicted embodiment, delivery catheter 300 has been removed and iliac limb 108 has been deployed. The iliac limb segment 108 of stent graft 100 seals the aneurysm sac at the proximal end 122. Injection catheter 302 has also been retracted so that injection ports 304 and 306 are within the aneurysmal sac 104. Polymeric micelles 308 can then be injected between the vessel lumen wall and the stent graft within the aneurysm sac 104 (FIG. 6c). The injection catheter 302 is then retrieved.

In another embodiment, a single lumen injection catheter can be used in the place of a multilumen injection catheter. After the guide wire is retrieved from the lumen, polymeric micelles can be delivered to the treatment site through the same lumen of the single lumen injection catheter. In an alternate embodiment, more than one single lumen injection catheter can be deployed in each iliac artery with the distal ends of the catheters meeting in the aneurysm sac.

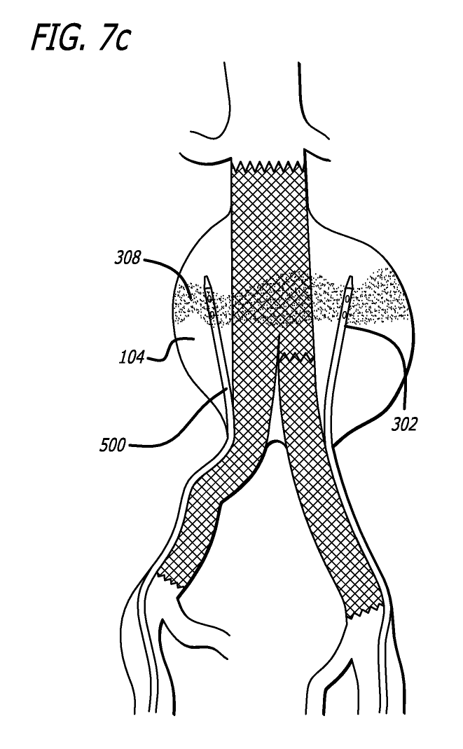

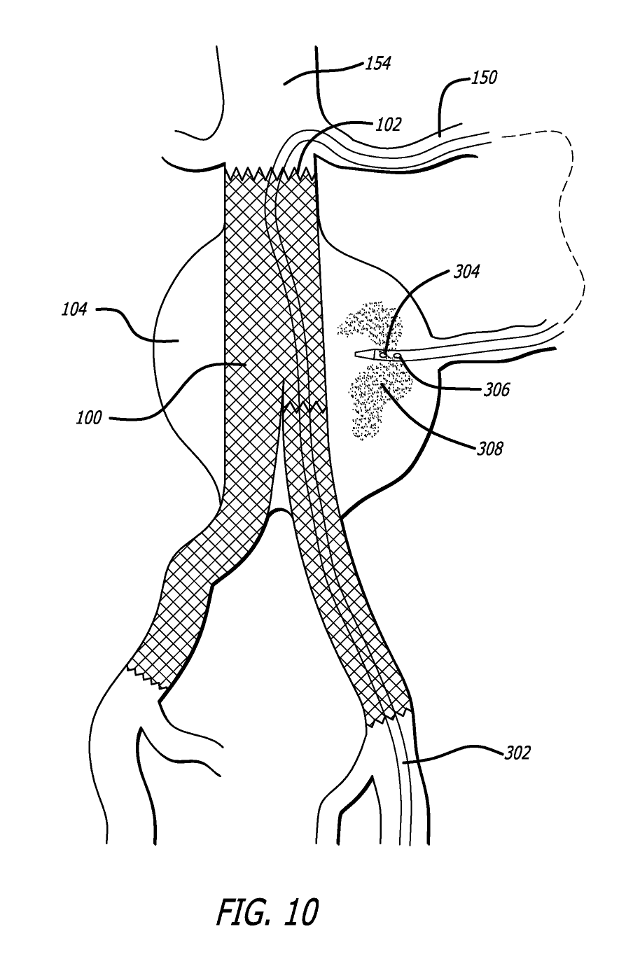

In another alternative embodiment, more than one injection catheter can be used to deliver polymeric micelles to the aneurysm sac (FIG. 7a). As previously described in FIGS. 1 and 6, stent graft 100 is deployed to the treatment site via the left iliac artery 114 (FIG. 7a). Multiple single lumen or multilumen injection catheters 302 and 500 are also deployed to the aneurysm sac 104 through the right iliac artery 116 and left iliac artery 114 (FIG. 10a). Injection catheters 302 and 500 have injection ports through which polymeric micelles can be deposited. Delivery catheter 300 is removed with both stent graft limbs deployed as in FIG. 7b while injection catheters 302 and 500 remain in place with their injection ports 304 and 306 and 504 and 506 in aneurysm sac 104. The iliac limb segment 108 of stent graft 100 seals the aneurysm sac at the proximal end 122 polymeric micelles 308 are then administered to the aneurysm sac 104 (FIG. 7c) and the injection catheters 302 and 500 can then be retrieved.

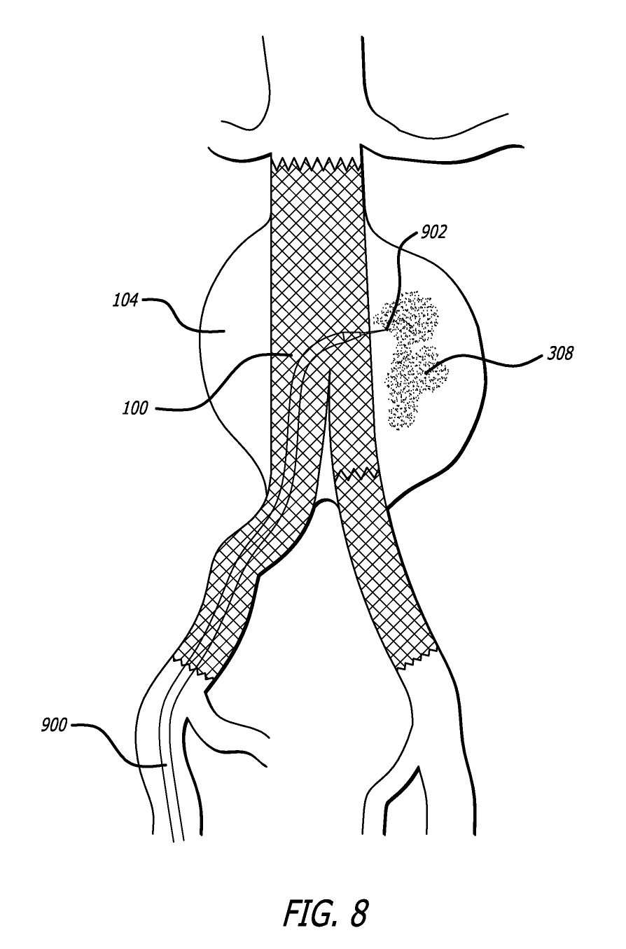

In yet another embodiment, polymeric micelles can be delivered to the aneurysm sac 104 by injecting the components through the wall of stent graft 100 (FIG. 8). Injection catheter 900 is advanced to the site of an already deployed stent graft 100 and needle 902 penetrates stent graft 100 to deliver polymeric micelles 308 to the aneurysm sac 104. Injection catheter 900 can be a multi-lumen or single lumen catheter.

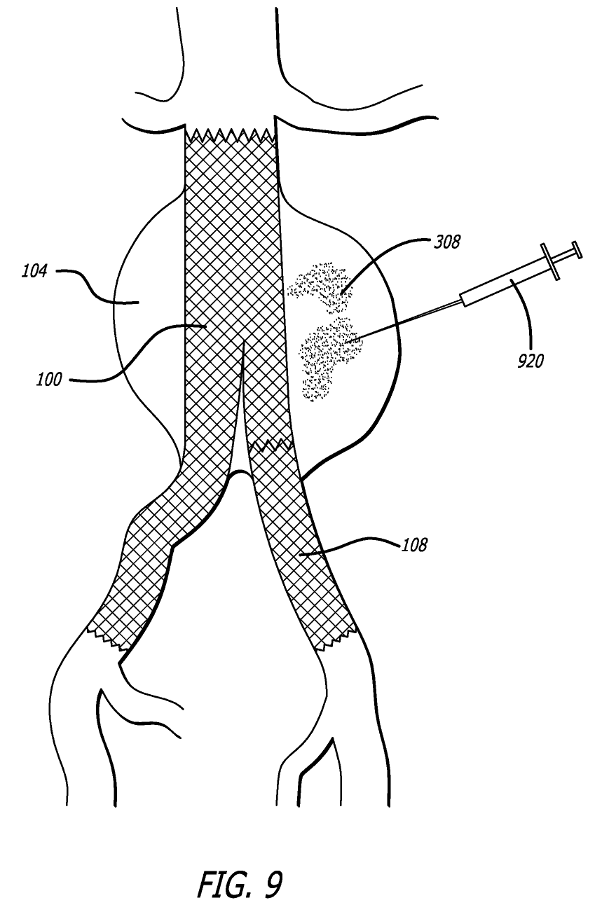

In another embodiment, polymeric micelles are delivered to the aneurysm sac 104 by translumbar injection (FIG. 9). Injection means 920, such as but not limited to a syringe, is directed, under radiographic or echographic guidance, to the aneurysm sac where stent graft 100 and iliac leg 108 have already been deployed. Injection means 920 delivers which polymeric micelles 308 to the aneurysm sac 104. Injection means 920 can have a single lumen or multiple lumens.

In yet another embodiment, depending on aneurysm location and stent graft placement, a collateral artery can be used to access the aneurysm sac (FIG. 10). For example, and not intended as a limitation, stent graft 100 can be deployed such that the distal end 102 is in the abdominal aorta 154 near, but below the renal artery. After deployment of stent graft 100, the deployment catheter is removed and an injection catheter 302 is advanced up the aorta past the aneurysm sac 104 to the superior mesenteric artery 150. The injection catheter 302 is then advanced through the superior mesenteric artery 150 and down into the inferior mesenteric artery where it originates at the aorta within aneurysm sac 104. The polymeric micelles 308 can then be injected into the aneurysm sac 104 through injection ports 304, 306.

Zinc chelator(s) are one class of compounds that can be used as therapeutic agents with the treatment methods described herein. As used herein, zinc chelator(s) include any compound that binds zinc (whether or not the molecule is a true chelator). Accordingly, any molecule that has the ability to ligand or chelate a zinc molecule (without making any deleterious electrostatic interactions) can be used with the present invention. Particular chelators that can be used include but are not limited to, histidine, spironaphthoxazine, EDTA (ethylenediamine tetraacetic acid), TPEN (tetrakis-(2-pyridylmethyl)ethylenediamine), EGTA (ethyleneglycol tetraacetic acid), DTPA (diethylenetriamine pentaacetic acid), CDTA (1,2-cyclohexanediaminetetraacetic acid), HEDTA (N-hydroxyethyl-ethylenediamine-triacetic acid), NTA (nitrilotriacetic acid), diacetic acid, hydroxamic acid, carboxylic acid, sulphydryl, or oxygenated phosphorus (for example, phosphinic acid and phosphonamidate, including aminophosphonic acid), citric acid, salicylic acid, malic acid, thiolate, polyphenols and flavonoids. Other useful compounds include those described in US Patent Application Publication Nos. 2008/0021024 and 2003/0073808, the entire contents of each of which are incorporated by reference herein.

Other non-limiting examples of therapeutic agents that can be used with the treatment methods disclosed herein include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, matrix metalloproteinase inhibitors and transforming nucleic acids. Therapeutic agents can also include anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like. Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487, the entire contents of which are incorporated by reference herein) and zotarolimus (ABT-578; see U.S. Pat. Nos. 6,015,815 and 6,329,386 the entire contents of each of which are incorporated by reference herein). Additionally, other rapamycin hydroxyesters as disclosed in U.S. Pat. No. 5,362,718, the entire contents of which are incorporated by reference herein, may be used.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained, each numerical parameter should at least be construed in light of the number of reported significant digits and by applying ordinary rounding techniques. Notwithstanding that the numerical ranges and parameters set forth are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the instant context are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate embodiments according to the invention and does not pose a limitation on the scope of the invention otherwise claimed. No language in the specification should be construed as presenting as essential any non-claimed element.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification should be deemed to contain the group as modified thus fulfilling the written description of all Markush groups used in the appended claims.

Certain embodiments are described herein. Of course, variations on these described embodiments will become apparent to those of ordinary skill in the art upon reading the foregoing description. The inventor expects skilled artisans to employ such variations as appropriate, even though they may be otherwise than specifically described herein. Moreover, any combination of the above-described elements in all possible variations thereof are to be considered encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles described. Modifications may be employed. Thus, by way of example, but not of limitation, alternative configurations of the present invention may be utilized in accordance with the teachings herein. Accordingly, embodiments according to the present invention are not limited to that precisely as shown and described.

What is claimed is:

1. A method of treating an aneurysm comprising:
delivering a stent graft to the site of said aneurysm;
deploying said stent graft to span said aneurysm;
locally administering polymeric micelles containing at least one therapeutic agent to said site of said aneurysm; and
exposing said polymeric micelles to sufficient energy to affect the release of said at least one therapeutic agent from said polymeric micelles.

2. The method according to claim 1 wherein said energy is selected from the group consisting of ultrasonic energy, radiofrequency (RF) energy, ultraviolet (UV) energy, infrared (IR) energy, visible light, and combinations thereof.

3. The method according to claim 1 wherein said locally administering comprises:
applying said polymeric micelles to the outer surface of said stent graft and/or
incorporating said polymeric micelles into a coating on said stent graft.

4. The method according to claim 1 wherein said locally administering comprises:
incorporating said polymeric micelles into a coating and
placing said coating on the outer surface of said stent graft.

5. The method according to claim 1 wherein said locally administering comprises:
attaching a delivery device to said stent graft wherein said delivery device holds and releases said polymeric micelles.

6. The method according to claim 4 wherein said delivery device is a pouch.

7. The method according to claim 1 wherein said locally administering comprises:
providing a stent graft with two layers;
partially adhering the layers together so that pouches are formed;
loading said pouches with polymeric micelles wherein following stent graft deployment the first layer is exposed to blood flow and the second layer faces the blood vessel wall and is semi-permeable.

8. The method according to claim 1 wherein said locally administering comprises:
applying said polymeric micelles directly to the outer surface of said stent graft while said stent graft is compressed within a stent deployment catheter.

9. The method according to claim 1 wherein said locally administering comprises:
administering said polymeric micelles through a delivery catheter and/or an injection catheter.

10. The method according to claim 8 wherein said polymeric micelles substantially fill the aneurysm sac.

11. The method according to claim 8 wherein said injection catheter is selected from the group consisting of a single lumen injection catheter and a multilumen injection catheter.

12. The method according to claim 8 comprising:
administering said polymeric micelles through at least two injection catheters wherein the first and second injection catheters reach said aneurysm through a different route.

13. A stent graft comprising polymeric micelles that contain at least one therapeutic agent and release said at least one therapeutic agent when exposed to energy wherein said polymeric micelles are one or more of applied to the outer surface of said stent graft, incorporated within a coating applied to said stent graft or within a delivery device associated with said stent graft.

14. The stent graft according to claim 13 wherein said stent graft comprises polymeric micelles incorporated within a coating applied to said stent graft wherein said coating is biodegradable.

15. The stent graft according to claim 13 wherein said stent graft comprises polymeric micelles incorporated within a coating applied to said stent graft wherein said coating is temperature-sensitive and/or ph-sensitive.

16. The stent graft according to claim 13 wherein said stent graft comprises polymeric micelles incorporated within a coating applied to said stent graft wherein said coating is formulated to be a quick-release coating, a medium-release coating or a slow-release coating.

17. The stent graft according to claim 13 wherein said delivery device is a pouch associated with said stent graft.

18. The stent graft according to claim 17 wherein said pouch is created by
providing a stent graft with two layers;
partially adhering the layers together so that one or more pouches are formed wherein following deployment the first layer is exposed to blood flow and the second layer faces the blood vessel wall and yer is semi-permeable.

19. An aneurysm treatment kit comprising:
a stent graft;
a stent graft delivery catheter;
polymeric micelles containing at least one therapeutic agent; and
an energy source.

20. The kit according to claim 19 wherein said energy source is selected from the group consisting of an ultrasonic energy source, a radiofrequency (RF) energy sources, an ultraviolet (UV) energy source, an infrared (IR) energy source, and a visible light source.
